(19) United States
(12) Patent Application Publication
(10) Pub. No.: US 2010/0068160 A1
(43) Pub. Date: Mar. 18, 2010

(54) COSMETIC AND DERMATOLOGICAL FORMULATIONS INCLUDING PHENOXYALKYL ESTERS

(75) Inventors: Oliver Springer, Wesel (DE); Klaus Jenni, Essen (DE)

Correspondence Address: SCULLY SCOTT MURPHY & PRESSER, PC, 400 GARDEN CITY PLAZA, SUITE 300, GARDEN CITY, NY, 11530, US

(73) Assignee: EVONIK GOLDSCHMIDT GMBH, Essen (DE)

(21) Appl. No.: 12/556,954

(22) Filed: Sep. 10, 2009

(30) Foreign Application Priority Data
Sep. 17, 2008 (DE) 102008042149.9

(51) Int. Cl. A61K 8/37; C07C 69/00; C07C 69/74; A61Q 17/04; A61K 8/26; A61K 8/27; A61K 8/28; A61K 47/14

(52) U.S. Cl. 424/59; 560/254; 560/122; 424/68; 424/67; 424/66; 514/785

(57) ABSTRACT

The invention relates to the use of phenoxyalkyl esters of C₆-C₁₂ alkyl- and cycloalkyl-carboxylic acids for the preparation of a cosmetic, dermatological or pharmaceutical formulation.

### FIELD OF THE INVENTION

The present invention relates to the use of phenoxyalkyl esters of C₆-C₁₂ alkyl- and cycloalkylcarboxylic acids for the preparation of a cosmetic, dermatological or pharmaceutical formulation. The phenoxyalkyl esters impart good sensory properties to the formulations and are characterized by very good dissolving properties for active ingredients, in particular organic UV photoprotective filters and deodorant or antiperspirant active ingredients.

### BACKGROUND OF THE INVENTION

In the field of cosmetic emulsions for skincare and haircare, a multitude of requirements is demanded by the consumer: apart from the cleaning and care effects, which determine the intended application. Emphasis is placed on such diverse parameters as highest possible dermatological compatibility, good refatting properties, elegant appearance, optimum sensory impression and storage stability.

Preparations which are used for the cleansing and care of the human skin and hair generally comprise a lipid and an aqueous phase besides a number of surface-active substances. As lipid phase/emollients are used, for example, hydrocarbons, ester oils and also plant and animal oils/fats/waxes. In order to satisfy the high requirements of the market with regard to sensory properties and optimum dermatological compatibility, new emollients and emulsifiers are continuously being developed and tested.

Furthermore, it is known that the ultraviolet part of natural and artificial light sources (UV-A 320-390 nm; UV-B 280-320 nm; UV-C 100 or 200-280 nm) in relatively large amounts leads to damage of the human skin.

UV-A radiation primarily brings about skin ageing (thinning of the epidermis and degeneration of connective tissue, pigment disorders), whereas UV-B and UV-C lead to sunburn and skin cancer.

Leisure time behavior, which has changed particularly in recent years, with prolonged stays outside and, in particular, extensive sunbathing to achieve a “healthy tan”, however, has, against the background of medical findings, and the awareness of a lack of natural protective mechanisms of the skin through pigment formation and sun habituation through thickening of the horny layer, pushed the need for adequate protection against intense UV radiation far into the foreground. It has been considerably reinforced by the discussion about the decrease and thinning of the Antarctic ozone layer and the associated increase in the intensity of UV-A and UV-B radiation on the Earth's surface.

This becomes clear from the increasing turnovers of products with high sun protection factors (SPF) in recent years. These are primarily still the classic sunscreen for emulations (sun milk, sun oil) with the primary intended use for sunbathing, although increasingly so-called care products for face, body and hair, such as day and night creams, conditioners, lotions, (hydro-, lipo)gels, (lip)sticks and sprays, pharmaceutical formulations and to a lesser extent, products of decorative cosmetics, which are available commercially predominantly in the form, of oils and liquid, creamy or salvy/pasty W/O and O/W emulsions.

The light protection factor LSF or SPF is a coefficient which expresses the ability of a product to prevent sunburn by the sun. Light protection with a factor of 60 therefore protects for twice as long as a product with factor 30 and accordingly three times as long as a product with factor 20 before sunburn occurs.

These relatively high light protection factors are in most cases produced by increasing the concentration of UV light protection filters in the formulation.

Since 1995, the light protection factors have been measured according to the same international standard (COLIPA), which permits a comparison between the products of different manufacturers.

In the case of frequent applications over large areas, it is not ruled out that the highly concentrated filters (ca. 3 to 30% by weight of the formulation) are applied to the skin in gram amounts. However, these amounts of filter substances have to have been dissolved and incorporated in the formulation in a homogeneous and stable manner.

To dissolve these substances, use is often made of oil-like components which have a good dissolving power for the filter substances. Thus, certain ester oils are, inter alia, used. One compound class used are aliphatic benzoic acid esters. A typical representative of this compound class is the compound C₁₂-C₁₅ alkyl benzoate which is used often as a solvent for UV photoprotective filters.

To protect against UV-B radiation, numerous compounds are known; these are mostly derivatives of 3-benzylidene camphor, of 4-aminobenzoic acid, of cinnamic acid, of salicylic acid, of benzophenone and also of 2-phenylbenzimidazole.

Also for the range between about 320 nm and about 400 nm, the so-called UV-A region, it is important to have available filter substances since its rays can also cause damage. Thus, it has been proven that UV-A radiation leads to damage of the elastic and collagenous fibres of connective tissue, which cause the skin to age prematurely, and that it is to be regarded as a cause of numerous phototoxic and photoallergic reactions. The harmful influence of UV-B radiation can be intensified by UV-A radiation.

A UV-B filter is tris(2-ethylhexyl) 4,4′,4″-(1,3,5-triazine-2,4,6-triyltriimino)trisbenzoate, synonymously: 2,4,6-tris[anilino(p-carbo-2′-ethyl-1′-hexyloxy)]-1,3,5-triazine, INCI name: Ethylhexyl Triazone. This UV-B filter substance is sold by BASF AG under the trade name UVINUL® T 150 and is characterized by having good UV absorption properties and good photostability.

The main disadvantage of this UV-B filter is the poor solubility in lipids. Customary solvents such as, for example, isopropyl palmitate, dicaprylyl ether, isohexadecane, butylene glycol dicaprylate/dicaprate can dissolve at most ca. 10% by weight of this filter, corresponding to about 1-1.5% by weight of dissolved, and thus active, UV photoprotective filter substance in the final formulation.

DE 25 19 433 describes 2-phenoxyethyl esters for use as soil fungi inhibitors.

Furthermore, phenoxyethyl esters are described as potential plasticizers for PVC in the journals Plasticheskie Massy (1974), volume 5, pages 58-60 and Azerbaidzhanskii Khimicheskii Zhurnal (1968), volume 6, pages 122-123.

Phenoxyethyl isobutyric acid ester (abbreviation: PEIB, CAS No. 103-60-6) is used as fragrance and food ingredient and has the FEMA No. 2873. This compound has been awarded the status GRAS (Generally Recognized as Safe) by the US Food and Drug Administration (FDA) and has thus received the safety approval for the food sector.

The preparation of phenoxyalkyl esters takes place by processes known to one skilled in the art. The esters can, for example, be prepared by esterifying phenoxyalkyl alcohols with C₆-C₁₂ alkyl- and cycloalkylcarboxylic acids. The preparation can also take place by transesterifying phenoxyalkyl alcohols with C₆-C₁₂ alkyl- and cycloalkylcarboxylic acid esters, in particular methyl, ethyl or isopropyl esters.

Examples of phenoxyalkyl alcohols are 2-phenoxyethanol and phenoxypropanol. Commercially available phenoxypropanol is often a mixture of 1-phenoxy-2-propanol (CAS No. 770-35-4) and 2-phenoxy-1-propanol (CAS No. 4169-04-4).

Examples of C₆-C₁₂ alkyl- and cycloalkylcarboxylic acids are caproic acid (hexanoic acid), cyclopentanecarboxylic acid, 2-methylpentanoic acid, heptanoic acid, cyclohexanecarboxylic acid, caprylic acid (octanoic acid), 2-ethylhexanoic acid, sorbic acid, pelargonic acid (nonanoic acid), isononanoic acid, 3,5,5-trimethylhexanoic acid, capric acid, 2-propylheptanoic acid, isodecanoic acid, undecanoic acid, laurie acid (dodecanoic acid), 2-butyloctanoic acid or mixtures of these carboxylic acids.

The transesterification can be carried out with or without catalysts at temperatures between 100° C. and 300° C. Suitable catalysts are, in particular, Lewis acids or Brönsted acids. Particularly preferred catalysts are sulphonic acids, such as, for example, para-toluenesulphonic acid, tin compounds, such as, for example, tin oxalate, tin oxide, dibutyltin diacetate, dibutyltin oxide or dibutyltin laurate, and titanium compounds, such as, for example, tetrapropyl titanate, tetraisopropyl titanate or tetrabutyl titanate.

The odor of the products obtained by esterification or transesterification can be improved as required by customary processes by removing readily volatile constituents, usually by distillation, steam stripping or gas stripping and other methods of deodorization. For this, the products are treated at 50° C.-200° C. and 0 mbar to 1013 mbar. Adsorbents, as described e.g., in EP 1585801, can also be used for improving the odor.

The color can also, if appropriate, be improved through treatment with hydrogen peroxide, sodium chlorite, hypophosphorous acid and other methods known to one skilled in the art. Examples are described, inter alia, in U.S. Patent Application Publication No. 2005/0008586.

### SUMMARY OF THE INVENTION

According to the invention, the term “C₆-C₁₂ alkyl- and cycloalkylcarboxylic acids” encompasses all carboxylic acids which have a carbon number of greater than or equal to 6 and less than or equal to 12. Both aliphatic, saturated, mono- and polyunsaturated, linear, branched and cyclic carboxylic acids are encompassed. Examples are: caproic acid, cyclopentanecarboxylic acid, 2-methylpentanoic acid, heptanoic acid, cyclohexanecarboxylic acid, caprylic acid, 2-ethylhexanoic acid, sorbic acid, isononanoic acid, 3,5,5-trimethyl-hexanoic acid, capric acid, 2-propylheptanoic acid, isodecanoic acid, undecanoic acid, undecylenic acid, 2-butyloctanoic acid.

According to the invention, the term “derivative” is to be understood as meaning structurally closely related derivatives of a basic chemical structure which have the same structural elements, but carry different substituents.

The present invention provides an emollient which, besides good sensory properties such as color, odor and skin feel, also has good solubility for active substances, in particular for organic UV photoprotective filters, such as, for example, triazines, and also for deodorant and antiperspirant active ingredients.

Surprisingly, it has been found that phenoxyalkyl esters of C₆-C₁₂ alkyl- and cycloalkylcarboxylic acids meet these requirements.

The invention thus provides the use of phenoxyalkyl esters of C₆-C₁₂ alkyl- and cyclo-alkylcarboxylic acids for the preparation of a cosmetic, dermatological or pharmaceutical formulation.

The invention further provides cosmetic, dermatological or pharmaceutical formulations comprising phenoxyalkyl esters of C₆-C₁₂ alkyl- and cycloalkylcarboxylic acids.

### DETAILED DESCRIPTION OF THE INVENTION

The use according to the invention of phenoxyalkyl esters of C₆-C₁₂ alkyl- and cycloalkylcarboxylic acids and formulations comprising the phenoxyalkyl esters according to the invention of C₆-C₁₂ alkyl- and cycloalkylcarboxylic acids are described below by way of example without any intention to limit the invention to these exemplary embodiments. Where ranges, general formulae or compound classes are given below, these are intended to encompass not only the corresponding ranges or groups of compounds which are explicitly mentioned, but also all part ranges and part groups of compounds which can be obtained by removing individual values (ranges) or compounds. Where documents are cited in the context of the present description, then the intention is for their content to form part of the disclosure of the present invention in their entirety. If, in the context of the present invention, compounds such as, for example, organomodified polysiloxanes, are described which can have different units several times, then these may occur in random distribution (random oligomer) or arranged (block oligomer) in these compounds. Details relating to the number of units in such compounds are to be understood as meaning an average value, averaged over all of the corresponding compounds. Unless stated otherwise, all of the percent (%) stated are percent by mass.

The phenoxyalkyl ester of C₆-C₁₂ alkyl- and cycloalkylcarboxylic acids used in this application includes at least one substance of the general formula (I)

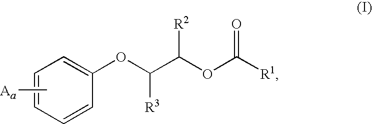

where R¹ is an unbranched or branched, saturated or unsaturated C₅₋₁₁ alkyl- or cycloalkyl radical,

R² and R³, independently of one another, identical or different, are hydrogen or methyl and

Aₐ where a=1-5, independently of one another, identical or different, are selected from the group: H, F, Cl, alkyl or fluoroalkyl radical, CN, CO₂R⁴, OH, OR⁵, O₂CR⁶, NR⁷R⁸, NO₂, SO₃R⁹, where R⁴ to R⁹, independently of one another, identical or different, are selected from the group: H, alkyl or fluoroalkyl radical.

Preference is given to using substances of the general formula (I) where A, independently of the others, identical or different, is selected from the group: H, F, alkyl radical, CN, OH, OR^{S}, where the alkyl radical is particularly preferably methyl and ethyl.

Furthermore, preference is given to using substances of the general formula (I) where R⁴ to R⁹, independently of one another, identical or different, are H or alkyl radical, where the alkyl radical is preferably methyl, ethyl, n-propyl, isopropyl, n-butyl, s-butyl, isobutyl, tert-butyl, and is particularly preferably methyl and ethyl.

Preference is given to using substances of the general formula (I) where a=1, particularly preferably where a=1 and A=H.

Preferably, the group OOC—R¹ of the acid radical of the acids is selected from the group caproic acid, cyclopentanecarboxylic acid, 2-methylpentanoic acid, heptanoic acid, cyclohexanecarboxylic acid, caprylic acid, 2-ethylhexanoic acid, sorbic acid, pelargonic acid, isononanoic acid, 3,5,5-trimethylhexanoic acid, capric acid, 2-propylheptanoic acid, isodecanoic acid, undecanoic acid, undecylenic acid and 2-butyloctanoic acid, and mixtures comprising at least two of these carboxylic acids, with caprylic acid, cyclohexanecarboxylic acid and pelargonic acid being particularly preferred.

Preferred combinations of R² and R³ are selected from the group

R²═R³═H,

R²═H, R³=methyl,

R²=methyl, R³═H, and combinations of these,

where R²═R³═H is particularly preferred.

These radicals are determined by the phenoxyalkyl alcohols used in the synthesis of the phenoxyalkyl esters, such as, for example 2-phenoxyethanol and phenoxypropanol. Commercially available phenoxypropanol is often a mixture of 1-phenoxy-2-propanol (CAS No. 770-35-4) and 2-phenoxy-1-propanol (CAS No. 4169-04-4).

Particularly preferred compounds of the general formula (I) are:

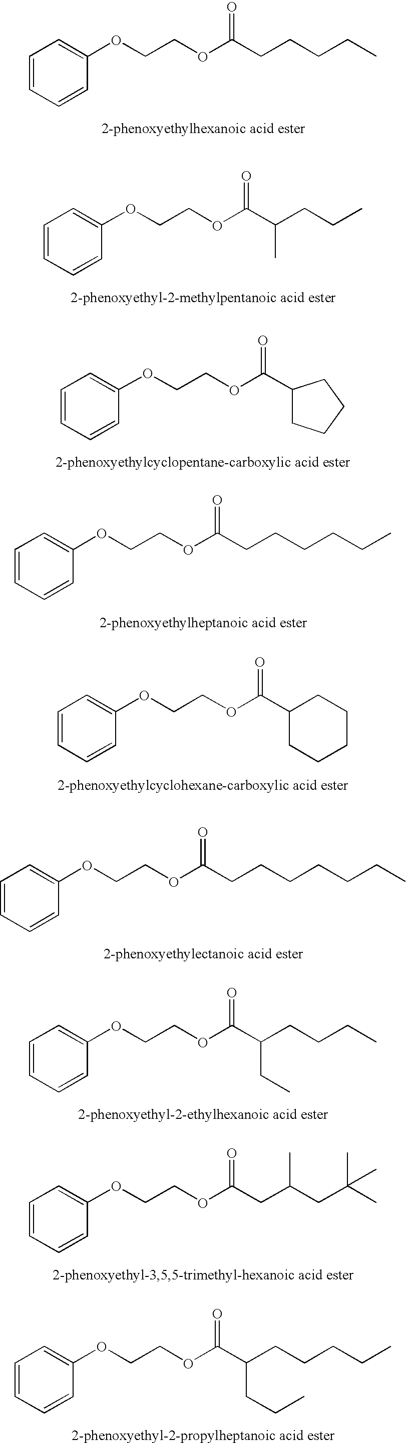

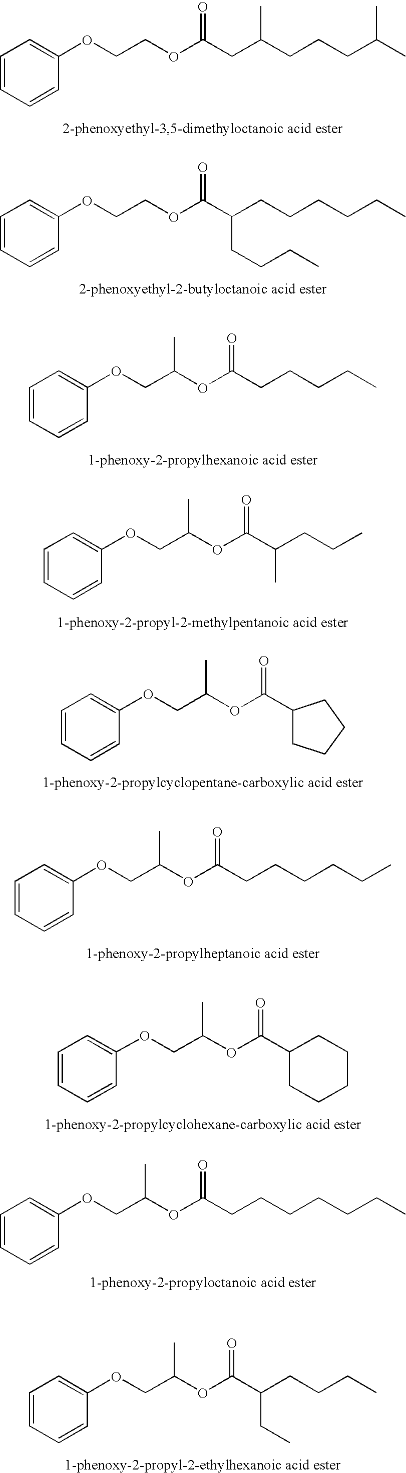

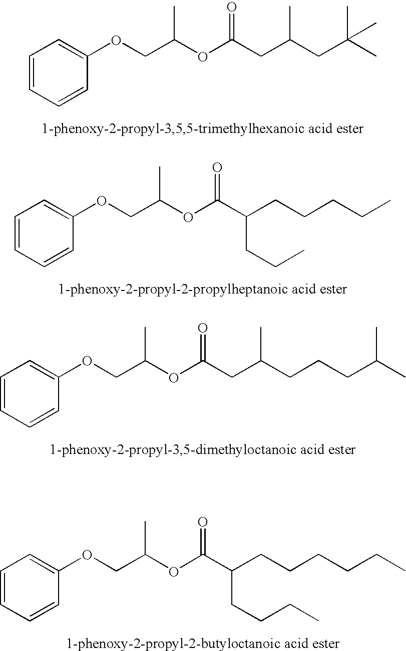

According to the invention, at least one substance of the general formula (I) is used for the preparation of a cosmetic, dermatological or pharmaceutical formulation, in particular of sunscreen preparations or deodorant/antiperspirant preparations.

Preferably, mixtures of the phenoxyalkyl esters can also be used.

The cosmetic, dermatological or pharmaceutical formulations can, for example, comprise at least one additional component selected from the group of

emollients,

emulsifiers and surfactants,

thickeners/viscosity regulators/stabilizers,

UV photoprotective filters,

antioxidants and vitamins,

hydrotropes (or polyols),

solids and fillers,

film formers,

pearlescent additives,

deodorant and antiperspirant active ingredients,

insect repellents,

self-tanning agents,

preservatives,

conditioners,

perfumes,

dyes,

biogenic active ingredients,

care additives,

superfatting agents,

solvents.

Emollients which can be used are all cosmetic oils, in particular mono- or diesters of linear and/or branched mono- and/or dicarboxylic acids having 2 to 44 carbon atoms with linear and/or branched saturated or unsaturated alcohols having 1 to 22 carbon atoms. It is also possible to use the esterification products of aliphatic, difunctional alcohols having 2 to 36 carbon atoms with monofunctional aliphatic carboxylic acids having 1 to 22 carbon atoms. Also suitable are long-chain aryl acid esters, such as, for example, esters of benzoic acid, e.g., benzoic acid esters of linear or branched, saturated or unsaturated alcohols having 1 to 22 carbon atoms, or else isostearyl benzoate or octyldodecyl benzoate. Further monoesters suitable as emollients and oil components are, for example, the methyl esters and isopropyl esters of fatty acids having 12 to 22 carbon atoms, such as, for example, methyl laurate, methyl stearate, methyl oleate, methyl erucate, isopropyl palmitate, isopropyl myristate, isopropyl stearate, isopropyl oleate. Other suitable monoesters are, for example, n-butyl stearate, n-hexyl laurate, n-decyl oleate, isooctyl stearate, isononyl palmitate, isononyl isononanoate, 2-ethylhexyl pahnitate, 2-ethylhexyl laurate, 2-hexyldecyl stearate, 2-octyldodecyl palmitate, oleyl oleate, oleyl erucate, erucyl oleate, and esters which are obtainable from technical-grade aliphatic alcohol cuts and technical-grade, aliphatic carboxylic acid mixtures, e.g., esters of unsaturated fatty alcohols, having 12 to 22 carbon atoms and saturated and unsaturated fatty acids having 12 to 22 carbon atoms, as are accessible from animal and vegetable fats. Also suitable are naturally occurring monoester and/or wax ester mixtures, as are present, for example in jojoba oil or in sperm oil. Suitable dicarboxylic acid esters are, for example, di-n-butyl adipate, di-n-butyl sebacate, di(2-ethylhexyl) adipate, di(2-hexyldecyl) succinate, diisotridecyl azelate. Suitable diol esters are, for example, ethylene glycol dioleate, ethylene glycol diisotridecanoate, propylene glycol di(2-ethylhexanoate), butanediol diisostearate, butanediol dicaprylate/caprate and neopentyl glycol dicaprylate. Further fatty acid esters which can be used as emollients are, for example, C₁₂-₁₅ alkyl benzoate, dicaprylyl carbonate, diethylhexyl carbonate. Emollients and oil components which can also be used are longer-chain triglycerides, i.e., triple esters of glycerol with three acid molecules, of which at least one is relatively long-chain. By way of example, mention may be made here of fatty acid triglycerides; examples of such which may be used are natural, vegetable oils, e.g., olive oil, sunflower oil, soybean oil, peanut oil, rapeseed oil, almond oil, sesame oil, avocado oil, castor oil, cocoa butter, palm oil, but also the liquid fractions of coconut oil or of palm kernel oil, and also animal oils, such as, for example, shark liver oil, cod liver oil, whale oil, beef tallow and butter fat, waxes such as beeswax, carnauba palm wax, spermaceti, lanolin and claw oil, the liquid fractions of beef tallow and also synthetic triglycerides of caprylic/capric acid mixtures, triglycerides of technical-grade oleic acid, triglycerides with isostearic acid, or from palmitic acid/oleic acid mixtures as emollients and oil components. Furthermore, hydrocarbons, in particular liquid paraffins and isoparaffins, can be used. Examples of hydrocarbons which can be used are paraffin oil, isohexadecane, polydecene, vaseline, Paraffinum perliquidum, squalane, ceresine. Furthermore, it is also possible to use linear or branched fatty alcohols such as oleyl alcohol or octyldodecanol, and also fatty alcohol ethers such as dicaprylyl ether. Suitable silicone oils and silicone waxes are, for example, polydimethylsiloxanes, cyclomethylsiloxanes, and also aryl- or alkyl- or alkoxy-substituted polymethylsiloxanes or cyclomethylsiloxanes. Suitable further oil bodies are, for example, Guerbet alcohols based on fatty alcohols having 6 to 18, preferably 8 to 10, carbon atoms, esters of linear C₆-C₂₂-fatty acids with linear C₆-C₂₂-fatty alcohols, esters of branched C₆-C₁₃-carboxylic acids with linear C6-C22-fatty alcohols, esters of linear C6-C22-fatty acids with branched C₈-C₁₈-alcohols, in particular 2-ethylhexanol or isononanol, esters of branched C₆-C₁₃-carboxylic acids with branched alcohols, in particular 2-ethylhexanol or isononanol, esters of linear and/or branched fatty acids with polyhydric alcohols (such as, for example, propylene glycol, dimerdiol or trimertriol) and/or Guerbet alcohols, triglycerides based on C₆-C₁₀-fatty acids, liquid mono-/di-/triglyceride mixtures based on C₆-C₁₈-fatty acids, esters of C₆-C₂₂-fatty alcohols and/or Guerbet alcohols with aromatic carboxylic acids, in particular benzoic acid, vegetable oils, branched primary alcohols, substituted cyclohexanes, linear C₆-C₂₂-fatty alcohol carbonates, Guerbet carbonates, esters of benzoic acid with linear and/or branched C6-C22-alcohols (e.g. Finsolv™ TN), dialkyl ethers, ring-opening products of epoxidized fatty acid esters with polyols, silicone oils and/or aliphatic or naphthenic hydrocarbons.

Emulsifiers or surfactants which may be used are nonionic, anionic, cationic or amphoteric surfactants.

Nonionic emulsifiers or surfactants which can be used are compounds from at least one of the following groups:
addition products of from 2 to 100 mol of ethylene oxide and/or 0 to 5 mol of propylene oxide onto linear fatty alcohols having 8 to 22 carbon atoms, onto fatty acids having 12 to 22 carbon atoms and onto alkylphenols having 8 to 15 carbon atoms in the alkyl group,
C_{12/18}-fatty acid mono- and diesters of addition products of from 1 to 100 mol of ethylene oxide onto glycerol,
glycerol mono- and diesters and sorbitan mono- and diesters of saturated and unsaturated fatty acids having 6 to 22 carbon atoms and ethylene oxide addition products thereof,
alkyl mono- and oligoglyco sides having 8 to 22 carbon atoms in the alkyl radical and ethylene oxide addition products thereof,
addition products of from 2 to 200 mol of ethylene oxide onto castor oil and/or hydrogenated castor oil,
partial esters based on linear, branched, unsaturated or saturated C₆-C₂₂-fatty acids, ricinoleic acid, and 12-hydroxystearic acid and glycerol, polyglycerol, pentaerythritol, dipentaerythritol, sugar alcohols (e.g., sorbitol), alkyl glucosides (e.g., methyl glucoside, butyl glucoside, lauryl glucoside) and polyglucosides (e.g., cellulose),
mono-, di- and trialkylphosphates, and mono-, di- and/or tri-PEG alkyl phosphates and salts thereof,
polysiloxane-polyether copolymers (dimethicone copolyols), such as, for example PEG/PPG-20/6 dimethicone, PEG/PPG-20/20 dimethicone, bis-PEG/PPG-20/20 dimethicone, PEG-12 or PEG-14 dimethicone, PEG/PPG-14/4 or 4/12 or 20/20 or 18/18 or 17/18 or 15/15,
polysiloxane-polyalkyl-polyether copolymers and corresponding derivatives, such as, for example, lauryl or cetyl dimethicone copolyols, in particular cetyl PEG/PPG-10/1 dimethicone (ABIL® EM 90 (Evonik Goldschmidt GmbH)),
mixed esters of pentaerythritol, fatty acids, citric acid and fatty alcohol as in DE 11 65 574 and/or mixed esters of fatty acids having 6 to 22 carbon atoms, methylglucose and polyols, such as, for example, glycerol or polyglycerol,
citric acid esters, such as, for example, glyceryl stearate citrate, glyceryl oleate citrate and dilauryl citrate.

Anionic emulsifiers or surfactants can contain water-solubilizing anionic groups, such as, for example, a carboxylate, sulphate, sulphonate or phosphate group and a lipophilic radical. Skin-compatible anionic surfactants are known to one skilled in the art in large numbers and are commercially available. Here, these may be alkyl sulphates or alkyl phosphates in the form of their alkali metal, ammonium or alkanolammonium salts, alkyl ether sulphates, alkyl ether carboxylates, acyl sarcosinates, and sulphosuccinates and acyl glutamates in the form of their alkali metal or ammonium salts.

Cationic emulsifiers and surfactants can also be added. Those which can be used are, in particular, quaternary ammonium compounds, in particular those provided with at least one linear and/or branched, saturated or unsaturated alkyl chain having 8 to 22 carbon atoms, such as, for example, alkyltrimethylammonium halides, such as, for example, cetyltrimethylammonium chloride or bromide or behenyltrimethylammonium chloride, but also dialkyldimethylammonium halides, such as, for example, distearyldimethylammonium chloride.

Furthermore, monoalkylamidoquats such as, for example, palmit-amidopropyltrimethylammonium chloride or corresponding dialkylamidoquats, can be used

Furthermore, readily biodegradable quaternary ester compounds can be used; these may be quaternized fatty acid esters based on mono-, di- or triethanolamine. Furthermore, alkylguanidinium salts can be added as cationic emulsifiers.

Typical examples of mild, i.e., particularly skin-compatible, surfactants are fatty alcohol polyglycol ether sulphates, monoglyceride sulphates, mono- and/or dialkyl sulphosuccinates, fatty acid isethionates, fatty acid sarcosinates, fatty acid taurides, fatty acid glutamates, ether carboxylic acids, alkyl oligoglucosides, fatty acid glucamides, alkylamidobetaines and/or protein fatty acid condensates, the latter for example based on wheat proteins.

Furthermore, it is possible to use amphoteric surfactants, such as, for example, betaines, amphoacetates or amphopropionates, thus, for example, substances such as the N-alkyl-N,N-dimethylammonium glycinates, for example cocoalkyldimethylammonium glycinate, N-acylaminopropyl-N,N-dimethylammonium glycinates, for example cocoa-cylaminopropyldimethylammonium glycinate, and 2-alkyl-3-carboxymethyl-3-hydroxyethyl-imidazolines having in each case 8 to 18 carbon atoms in the alkyl or acyl group, and also cocoacylaminoethyl hydroxyethylcarboxymethyl glycinate.

Of the ampholytic surfactants, it is possible to use those surface-active compounds which, apart from a C₈/₁₈-alkyl or -acyl group in the molecule, contain at least one free amino group and at least one —COON or —SO₃H group and are capable of forming internal salts. Examples of suitable ampholytic surfactants are N-alkylglycines, N-alkylpropionic acids, N-alkylaminobutyric acids, N-alkyliminodipropionic acids, N-hydroxyethyl-N-alkylamido-propylglycines, N-alkyltaurines, N-alkylsarcosines, 2-alkylaminopropionic acids and alkylaminoacetic acids having in each case about 8 to 18 carbon atoms in the alkyl group. Further examples of ampholytic surfactants are N-cocoalkylaminopropionate, cocoacylaminoethylaminopropionate and C₁₂/₁₈-acylsarcosine.

Suitable thickeners are, for example, polysaccharides, in particular xanthan gum, guar guar, agar agar, alginates and tyloses, carboxymethylcellulose and hydroxyethylcellulose, also relatively high molecular weight polyethylene glycol mono- and diesters of fatty acids, polyacrylates (e.g., Carbopols™ or Synthalens™), polyacrylamides, polyvinyl alcohol and polyvinylpyrrolidone, surfactants such as, for example, ethoxylated fatty acid glycerides, esters of fatty acids with polyols, such as, for example, pentaerythritol or trimethyloipropane, fatty alcohol ethoxylates with a narrowed homologue distribution or alkyl oligoglucosides, and also electrolytes such as sodium chloride and ammonium chloride.

Suitable thickeners for thickening oil phases are all thickeners known to one skilled in the art. In particular, mention is to be made here of waxes, such as hydrogenated castor wax, beeswax or microwax. Furthermore, inorganic thickeners can also be used, such as silica, alumina or sheet silicates (e.g., hectorite, laponite, saponite). In this regard, these inorganic oil phase thickeners may be hydrophobically modified. For the thickening/stabilization of water-in-oil emulsions, in particular aerosils, sheet silicates and/or metal salts of fatty acids, such as, for example, magnesium stearate, aluminium stearate and/or zinc stearate, or magnesium ricinoleate, aluminium ricinoleate and/or zinc ricinoleate, can be used here.

Viscosity regulators for aqueous surfactant systems which may be present are, for example NaCl, low molecular weight nonionic surfactants, such as cocoamide DEA/MEA and laureth-3, or polymeric, high molecular weight, associative, highly ethoxylated fat derivatives, such as PEG-200 hydrogenated glyceryl palmate.

UV photoprotective filters which can be used are, for example, organic substances which are able to absorb ultraviolet rays and which give off the absorbed energy again in the form of longer-wave radiation, e.g., heat. UVB filters may be oil-soluble or water-soluble.

### Examples of oil-soluble UVB photoprotective filters are

3-benzylidenecamphor and derivatives thereof, e.g., 3-(4-methylbenzylidene)camphor,

4-aminobenzoic acid derivatives, such as, for example, 2-ethylhexyl 4-(dimethylamino)benzoate, 2-ethylhexyl 4-(dimethylamino)benzoate and amyl 4-(dimethylamino)benzoate,

esters of cinnamic acid, such as 2-ethylhexyl 4-methoxycinnamate, isopentyl 4-methoxycinnamate, 2-ethylhexyl 2-cyano-3-phenylcinnamate (octocrylene),

esters of salicylic acid, such as, for example, 2-ethylhexyl salicylate, 4-isopropylbenzyl salicylate, homomethyl salicylate,

derivatives of benzophenone, such as, for example, 2-hydroxy-4-methoxybenzophenone, 2-hydroxy-4-methoxy-4′-methylbenzophenone, 2,2′-dihydroxy-4-methoxybenzophenone, esters of benzalmalonic acid, such as, for example, di-2-ethylhexyl 4-methoxybenzmalonate,

triazine derivatives, such as, for example, 2,4,6-trianilino(p-carbo-2′-ethyl-octyltriazone and those described in EP 1180359 and DE 2004/02475,

propane-1,3-diones, such as, for example, 1-(4-tert-butylphenyl)-3-(4′-methoxyphenyl)propane-1,3-dione.

Suitable water-soluble UVB photoprotective filters are:

2-phenylbenzimidazole-5-sulphonic acid and the alkali metal, alkaline earth metal, ammonium, alkylammonium, alkanolammonium and glucammonium salts thereof,

sulphonic acid derivatives of benzophenone, such as, for example, 2-hydroxy-4-methoxybenzophenone-5-sulphonic acid and its salts,

sulphonic acid derivatives of 3-benzylidenecamphor, such as, for example, 4-(2-oxo-3-bornylidenemethyl)benzenesulphonic acid and 2-methyl-5-(2-oxo-3-bornylidene)sulphonic acid and salts thereof.

Suitable typical UVA photoprotective filters are in particular derivatives of benzoylmethane, such as, for example, 1-(4′-tert-butylphenyl)-3-(4′-methoxyphenyl)propane-1,3-dione or 1-phenyl-3-(4′-isopropylphenyl)propane-1,3-dione. The UV-A and UV-B filters can of course also be used in mixtures.

Besides the specified soluble substances, insoluble pigments, namely finely disperse metal oxides or salts are also suitable for this purpose, such as, for example, titanium dioxide, zinc oxide, iron oxide, aluminium oxide, cerium oxide, zirconium oxide, silicates (talc), barium sulphate and zinc stearate. The particles here should have an average diameter of less than 100 nm, e.g., between 5 nm and 50 nm and in particular between 15 nm and 30 nm. They can have a spherical shape, although it is also possible to use those particles which have an ellipsoidal shape or a shape which deviates in some other way from the spherical form. A relatively new class of photoprotective filters are micronized organic pigments, such as, for example, 2,2′-methylenebis {6-(2H-benzotriazol-2-yl)-4-(1,1,3,3-tetramethylbutyl)phenol} with a particle size of <200 nm, which is obtainable, for example, as 50% strength aqueous dispersion.

Further suitable UV photoprotective filters can be found in the overview by P. Finkel in SÖFW-Journal 122, 543 (1996).

Besides the two aforementioned groups of primary UV photoprotective filters, it is also possible to use secondary photoprotective agents of the antioxidant type which interrupt the photochemical reaction chain which is triggered when UV radiation penetrates into the skin.

Antioxidants and vitamins which can be used are, for example, superoxide-dismutase, tocopherol (vitamin E), tocopherol sorbate, tocopherol acetate, other esters of tocopherol, dibutylhydroxytoluene and ascorbic acid (vitamin C) and its salts, and also derivatives thereof (e.g., magnesium ascorbyl phosphate, sodium ascorbyl phosphate, ascorbyl sorbate), ascorbyl esters of fatty acids, butylated hydroxybenzoic acid and its salts, peroxides, such as, for example, hydrogen peroxide, perborates, thioglycolates, persulphate salts, 6-hydroxy-2,5,7,8-tetramethylchroman-2-carboxylic acid (TROLOX®), gallic acid and its alkyl esters, uric acid and its salts and alkyl esters, sorbic acid and its salts, lipoic acid, ferulic acid, amines (e.g., N,N-diethylhydroxylamine, aminoguanidines), sulfhydryl compounds (e.g., glutathione), dihydroxy-fumaric acid and its salts, glycine pidolate, arginine pidolate, nordihydroguaiaretic acid, bioflavonoids, curcumin, lysine, L-methionine, proline, superoxide dismutase, silymarin, tea extract, grapefruit peel/pip extract, melanin, rosemary extract, thiooctanoic acid, resveratrol, oxyresveratrol, etc.

Hydrotropes which can be used for improving the flow behavior and the application properties are, for example, ethanol, isopropyl alcohol or polyols. Polyols which are suitable here can have 2 to 15 carbon atoms and at least two hydroxyl groups. Typical examples are: glycerol alkylene glycols, such as, for example, ethylene glycol, diethylene glycol, propylene glycol, butylene glycol, hexylene glycol, and polyethylene glycols with an average molecular weight of from 100 to 1,000 daltons, technical-grade oligoglycerol mixtures with a degree of self-condensation of from 1.5 to 10, such as, for example, technical-grade diglycerol mixtures with a diglycerol content of from 40 to 50% by weight, methylol compounds, such as in particular trimethylolethane, trimethylolpropane, trimethylolbutane, pentaerythritol and dipentaerythritol, lower alkyl glucosides, in particular those with 1 to 4 carbon atoms in the alkyl radical, such as, for example, methyl and butyl glucoside, sugar alcohols having 5 to 12 carbon atoms, such as, for example, sorbitol or mannitol, sugars having 5 to 12 carbon atoms, such as, for example, glucose or sucrose, amino sugars, such as, for example, glucamine.

Solids which can be used are, for example, iron oxide pigments, titanium dioxide or zinc oxide particles and those additionally specified under “UV protectants”. Furthermore, it is also possible to use particles which lead to special sensory effects, such as, for example, nylon-12, boron nitride, polymer particles such as, for example, polyacrylate or polymethyl acrylate particles or silicone elastomers. Fillers which can be used include starch and starch derivatives, such as tapioca starch, distarch phosphate, aluminium starch or sodium starch, octenyl succinate, and pigments which have neither primarily a UV filter effect, nor a coloring effect, for example Aerosils® (CAS No. 7631-86-9).

Examples of film formers which can be used, for example, for improving the water resistance are: polyurethanes, dimethicones, copolyol, polyacrylates or PVP/VA copolymer (PVP=polyvinylpyrrolidone, VA=vinyl acetate). Fat-soluble film formers which can be used are: e.g., polymers based on polyvinylpyrrolidone (PVP), copolymers of polyvinylpyrrolidone, PVP/hexadecene copolymer or the PVP/eicosene copolymer.

Pearlescence additives which can be used are, for example, glycol distearates or PEG-3 distearate.

Suitable deodorant active ingredients are, for example, odor concealers such as the customary perfume constituents, odor absorbers, for example the sheet silicates described in the patent laid-open specification DE 40 09 347, of these, in particular montmorillonite, kaolinite, illite, beidelite, nontronite, saponite, hectorite, bentonite, smectite, or also, for example, zinc salts of ricinoleic acid. Antimicrobial agents are likewise suitable for being incorporated. Antimicrobial substances are, for example, 2,4,4′-trichloro-2′-hydroxydiphenyl ether (Irgasan), 1,6-di-(4-chlorophenylbiguanido)hexane (chlorhexidine), 3,4,4′-trichlorocarbonilide, quaternary ammonium compounds, clove oil, mint oil, thyme oil, triethyl citrate, farnesol (3,7,11-trimethyl-2,6,10-dodecatrien-1-ol), ethylhexyl glyceryl ether, polyglyceryl-3 caprylate (TEGO® Cosmo P813, Evonik Goldschmidt GmbH), and the effective agents described in the patent laid-open specifications DE 198 55 934, DE 37 40 186, DE 39 38 140, DE 42 04 321, DE 42 29 707, DE 42 29 737, DE 42 38 081, DE 43 09 372, DE 43 24 219 and EP 666 732.

Antiperspirant active ingredients which may be used are astringents, for example basic aluminium chlorides such as aluminium chlorohydrate (“ACH”) and aluminium zirconium glycine salts (“ZAG”).

Insect repellents which may be used are, for example, N,N-diethyl-m-toluamide, 1,2-pentanediol or Insect Repellent 3535.

Self-tanning agents which can be used are, for example, dihydroxyacetone and erythrulose.

Preservatives which can be used are, for example, mixtures of one or more alkyl paraben esters with phenoxyethanol. The alkyl paraben esters may be methyl paraben, ethyl paraben, propyl paraben and/or butyl paraben. Instead of phenoxyethanol, it is also possible to use other alcohols, such as, for example, benzyl alcohol or ethanol. Moreover, it is also possible to use other customary preservatives such as, for example, sorbic acid or benzoic acid, salicylic acid, 2-bromo-2-nitropropane-1,3-diol, chloroacetamide, diazolidinylurea, DMDM hydantoin, iodopropynyl butylcarbamate, sodium hydroxymethylglyeinates, methyl-isothiazoline, chloromethylisothiazoline, ethylhexylglycerol or caprylyl glycol.

Conditioning agents which can be used are, for example, organic quaternary compounds, such as cetrimonium chloride, dicetyldimonium chloride, behentrimonium chloride, distearyldimonium chloride, behentrimonium methosulphate, distearoylethyldimonium chloride, palmitamidopropyltrimonium chloride, guar hydroxypropyltrimonium chloride, hydroxypropylguar, hydroxypropyltrimonium chloride, or quaternium-80 or else amine derivatives such as, for example, aminopropyldimethicones or stearamidopropyldimethylamines.

Perfumes which can be used are natural or synthetic odorants or mixtures thereof. Natural odorants are extracts from flowers (lily, lavender, rose, jasmine, neroli, ylang ylang), stems and leaves (geranium, patchouli, petitgrain), fruits (anise, coriander, caraway, juniper), fruit peels (bergamot, lemon, orange), roots, (mace, angelica, celery, cardamon, costus, iris, thyme), needles and branches (spruce, fir, pine, dwarf-pine), resins and balsams (galbanum, elemi, benzoin, myrrh, olibanum, opoponax). Animal raw materials are also suitable, such as, for example, civet and eastoreum. Typical synthetic odorant compounds are products of the ester, ether, aldehyde, ketone, alcohol and hydrocarbon types. Odorant compounds of the ester type are, for example, benzyl acetate, phenoxyethyl isobutyrate, p-tert-butylcyclohexyl acetate, linalyl acetate, dimethylbenzylcarbinyl acetate, phenylethyl acetate, linalyl benzoate, benzyl formate, ethylmethylphenyl glycinate, allylcyclohexyl propionate, styrallyl propionate and benzyl salicylate. The ethers include, for example, benzyl ethyl ether, the aldehydes include, for example, the linear alkanals having 8 to 18 carbon atoms, citral, citronellal, citronellyloxyacetaldehyde, cyclamenaldehyde, hydroycitronellal, filial and bourgeonal, the ketones include, for example, the ionones, α-isomethylionone and methyl cedryl ketone, the alcohols include anethole, citronellol, eugenol, isoeugenol, geraniol, linalool, phenylethyl alcohol and terpineol, and the hydrocarbons include primarily the terpenes and balsams. It is possible to use mixtures of different odorants which together produce a pleasant scent note. Essential oils of low volatility, which are mostly used as aroma components, are also suitable as perfumes, e.g., sage oil, camomile oil, clove oil, melissa oil, mint oil, cinnamon leaf oil, linden blossom oil, juniper berry oil, vetiver oil, olibanum oil, galbanum oil, labolanum oil and lavandin oil. It is also possible to use bergamot oil, dihydromyrcenol, filial, lyral, citronellol, phenylethyl alcohol, α-hexyleinnamaldehyde, geraniol, benzyl acetone, cyclamenaldehyde, linalool, boisambrene forte, ambroxan, indole, hedione, sandelice, lemon oil, mandarin oil, orange oil, allyl amyl glycolate, cyclovertal, lavandin oil, clary sage oil, β-damascone, geranium oil bourbon, cyclohexyl salicylate, vertofix coeur, iso-E-super, fixolide NP, evernyl, iraldein gamma, phenylacetic acid, geranyl acetate, benzyl acetate, rose oxide, romillat, irotyl and floramat alone or in mixtures.

Dyes which can be used are the substances approved and suitable for cosmetic purposes, as are listed, for example, in the publication “Cosmetic Colourants” of the Dyes Commission of the German Research Society, Verlag Chemie, Weinheim, 1984, pp. 81 to 106. These dyes are usually used in concentrations of from 0.001 to 0.1% by weight, based on the total mixture.

Biogenic active ingredients are to be understood as meaning, for example, tocopherol, tocopherol acetate, tocopherol palmitate, ascorbic acid, polyphenols, deoxyribonucleic acid, coenzyme Q10, retinol, AHA acids, amino acids, hyaluronic acid, alpha-hydroxy acids, isoflavones, polyglutamic acid, creatine (and creatine derivatives), guanidine (and guanidine derivatives), pseudoceramides, essential oils, peptides, protein hydrolysates, plant extracts, bisabolol, allantoin, panthenol, phytantriol, idebenone, liquorice extract, glycyrrhizidine and idebenone, scleroglucan, β-glucan, santalbic acid and vitamin complexes. Examples of plant extracts are horsechestnut extract, camomile extract, rosemary extract, black and red currant extract, birch extract, rosehip extract, algae extract, green tea extract, aloe extract, ginseng extract, ginkgo extract, grapefruit extract, calendula extract, camphor, thyme extract, mangosteen extract, cystus extract, terminalia arjuna extract, oat extract, oregano extract, raspberry extract, strawberry extract, etc.

The biogenic active ingredients can also include the so-called barrier lipids, examples of which being ceramides, phytosphingosine and derivatives, sphingosine and derivatives, sphinganine and derivatives, pseudoceramides, phospholipids, lysophospholipids, cholesterol and derivatives, cholesteryl ester, free fatty acids, lanolin and derivatives, squalene, squalene and related substances.

Within the context of the invention, the biogenic active ingredients also include anti-acne, such as, for example, benzyl peroxide, phytosphingosine and derivatives, niacinamide hydroxybenzoate, nicotinaldehyde, retinol acid and derivatives, salicylic acid and derivatives, citronellic acid etc., and anti-cellulite, such as, for example, xanthine compounds such as caffeine, theophylline, theobromine and aminophylline, carnitine, carnosine, salicyloyl phytosphingosine, phytosphingosines, santalbic acid etc., as well as antidandruff agents such as, for example, salicylic acid and derivatives, zinc pyrithione, selenium sulphide, sulphur, cyclopiroxolamine, bifonazole, climbazole, octopirox and actirox etc., as well as astringents, such as, for example, alcohol, aluminium derivatives, gallic acid, pyridoxine salicylate, zinc salts, such as, for example, zinc sulphate, acetate, chloride, lactate, zirconium chlorohydrates etc.

Bleaches such as kojic acid, arbutin, vitamin C and derivatives, hydroquinone, turmeric oil, creatinine, sphingolipids, niacinamide, etc. may also be included in the biogenic active ingredients.

Care additives which may be present are, for example, ethoxylated glycerol fatty acid esters, such as, for example, PEG-7 glycerol cocoate, or cationic polymers, such as, for example, polyquaternium-7 or polyglycerol esters.

Superfatting agents which can be used are substances such as, for example, lanolin and lecithin, and also polyethoxylated or acylated lanolin and lecithin derivatives, polyol fatty acid esters, monoglycerides and fatty acid alkanolamides, with the latter simultaneously serving as foam stabilizers.

Solvents which can be used are, for example, aliphatic alcohols such as ethanol, propanol or 1,3-propanediol, cyclic carbonates, such as ethylene carbonate, propylene carbonate, glycerol carbonate, esters of mono- or polycarboxylic acids such as ethyl acetate, ethyl lactate, dimethyl adipate and diethyl adipate, propylene glycol, dipropylene glycol, glycerol, glycerol carbonate or water.

Use according to the invention is particularly suitable for producing sunscreen preparations.

The invention therefore further provides the use of phenoxyalkyl esters of C₆-C₁₂ alkyl- and cycloalkylcarboxylic acids of the general formula (I) in cosmetic, dermatological or pharmaceutical formulations as solubility promoters or solvents of at least one UV photoprotective filter substance.

When using phenoxyalkyl esters of C₆-C₁₂ alkyl- and cycloalkylcarboxylic acids of the general formula (I) as solubility promoters or solvents of UV photoprotective filter substances, preference is given to using those phenoxyalkyl esters which have been described in the case of the use of the groups of phenoxyalkyl esters described above, in particular the groups described above as preferred, for the preparation of the formulations.

Consequently, a preferably used additional component is also the group of substances of UV photoprotective filters. Here, preferably the lipophilic, hydrophobic UV photoprotective filter substances, in particular triazine derivatives, are used.

As UV-B filters, particular preference is given to using the photoprotective filter substances 2-ethylhexyl 2-cyano-3-phenylcinnamate, 2,4-bis{[4-(2-ethylhexyloxy)-2-hydroxy]phenyl}-6-(4-methoxyphenyl)-1,3,5-triazine, dioctylbutylamidotriazone, 2-hydroxy-4-methoxybenzophenone, 2-hydroxy-4-methoxy-4′-methylbenzophenone, 2,2′-dihydroxy-4-methoxybenzophenone, di-2-ethylhexyl 4-methoxybenzmalonate, 2,4,6-tris[anilino-(p-carbo-2′-ethyl-1′-hexyloxy)]-1,3,5-triazine, 2,4-bis[5,1(dimethylpropyl)benzoxazol-2-yl-(4-phenyl)imino]-6-(2-ethylhexyl)imino-1,3,5-triazine, 2,4-bis{[4-(2-ethyl-hexyloxy)-2-hydroxy]phenyl}-6-(4-methoxyphenyl)-1,3,5-triazine and 2-[4,6-bis(2,4-dimethylphenyl)-1,3,5-triazin-2-yl]-5-(octyloxy)phenol.

The UV-A filters used are preferably 1-(4′-tert-butylphenyl)-3-(4′-methoxyphenyl)propane-1,3-dione, 1-phenyl-3-(4′-isopropylphenyl)propane-1,3-dione.

Particularly preferred UV-A filters are 4-(tert-butyl)-4′-methoxydibenzoylmethane (CAS No. 70356-09-1), which is sold by Givaudan under the name Parsol® 1789 and by Merck under the tradename Eusolex® 9020, and hydroxybenzophenones according to DE 102004027475, particularly preferably the hexyl ester of 2-(4′-diethylamino-2′-hydroxybenzoyl)-benzoic acid (also: aminobenzophenone), which is available under the name Uvinul A Plus from BASF.

Furthermore preferred UV filter substances are also so-called broadband filters, i.e., filter substances which absorb both UV-A radiation and also UV-B radiation. Within this group, preference is given to using 2,2′-methylene-bis(6-(2H-benzotriazol-2-yl)-4-(1,1,3,3-tetramethylbutyl)-phenol, which is available under the tradename Tinosorb® M from CIBA-Chemikalien GmbH, and 2-(2H-benzotriazol-2-yl)-4-methyl-6-[2-methyl-3-[1,3,3,3-tetramethyl-1-[(trimethylsilyl)oxy]disiloxanyl]propyl]phenol (CAS No.: 155633-54-8) with the INCI name Drometrizole Trisiloxane.

The amount of the UV photoprotective filters used is preferably 0.01-15%, preferably 0.05-10%, particularly preferably 0.1-5%, based on the formulation.

Preference is given to using a combination of two or more different UV filters.

A further preferably used additional component is the group of film formers in order to improve the water resistance of the compositions and thus also to increase the UV protection performance. Preferably used film formers are polyurethanes, dimethicones, copolyol, polyacrylates, PVP/VA copolymer (PVP=polyvinylpyrrolidone, VA=vinyl acetate), polyvinylpyrrolidone (PVP), copolymers of polyvinylpyrrolidone, PVP/hexadecene copolymer or the PVP/eicosene copolymer.

The use according to the invention is also suitable for producing deodorant/antiperspirant preparations.

The invention also further provides the use of phenoxyalkyl esters of C₆-C₁₂ alkyl- and cycloalkylcarboxylic acids of the general formula (I) in cosmetic, dermatological or pharmaceutical formulations as solubility promoters or solvents of at least one deodorant or antiperspirant active ingredient.

When using phenoxyalkyl esters of C₆-C₁₂ alkyl- and cycloalkylcarboxylic acids of the general formula (I) as solubility promoters or solvents of deodorant or antiperspirant active ingredients, preference is given to using those phenoxyalkyl esters which have been described in the case of the use of the groups of phenoxyalkyl esters described above, in particular the groups described above as preferred, for the preparation of the formulations.

Consequently, a further preferably used additional component is the group of deodorant and antiperspirant active ingredients. From this group, preference is given to using astringents, particularly preferably aluminium salts, zinc salts or aluminium-zirconium complexes.

The invention moreover provides cosmetic, dermatological or pharmaceutical formulations comprising a substance of the general formula (I).

The cosmetic, dermatological or pharmaceutical formulations according to the invention are preferably obtained through the use of the groups of phenoxyalkyl esters described above, in particular the groups described above as preferred, for the preparation of the formulations and also for use as solubility promoters. The cosmetic, dermatological or pharmaceutical formulations preferably comprise further additional components as that these additional components are preferably used as described above for the use as solubility promoters.

Formulations according to the invention can, for example, be used as a skincare product, facecare product, headcare product, bodycare product, intimate care product, footcare product, haircare product, nailcare product, dental care product or mouth care product.

Formulations according to the invention can, for example, be used in the form of an emulsion, a suspension, a solution, a cream, a salve, a paste, a gel, an oil, a powder, an aerosol, a stick, a spray, a foam, a cleansing product, a make-up or sunscreen preparation or a face toner.

Preferably, the cosmetic, dermatological or pharmaceutical formulations comprise 0.1 to 60 percent by weight, preferably 1 to 25 percent by weight and particularly preferably 3 to 15 percent by weight, of at least one substance of the general formula (I).

Particularly preferred cosmetic, dermatological or pharmaceutical formulations comprise:
a) 0.1-60 percent by weight of at least one substance of the general formula (I),
b) 0.1-20 percent by weight of surfactants and/or emulsifiers and/or coemulsifiers,
e) 0.1-40 percent by weight of further oil bodies and
d) 0-98 percent by weight of water,
   where the weight percentages of components (a), (b), (c) and (d) add up to 100 percent by weight.

The present invention is described by way of example in the examples listed below, without any intention to limit the invention, the scope of application of which arises from the entire description and the claims, to the embodiments specified in the examples.

### Example 1

### Preparation of 2-phenoxyethyloctanoic acid ester

96.8 g (0.70 mol) of 2-phenoxyethanol and 112.2 g (0.78 mol) of caprylic acid were heated together with 1.1 g of para-toluenesulphonic acid for 6 h at 150° C.-160° C. on a water separator. Excess acid was distilled off in vacuo and the reaction mixture was cooled. The catalyst was neutralized with potassium hydroxide solution and the product was then bleached and deodorized with hydrogen peroxide. Following filtration, 176.5 g of an odorless oil are obtained: OHV<1; AV=0.1; color (APHA)=52 Hazen.

### Example 2

### Preparation of 2-phenoxyethyloctanoic acid ester

193.7 g (1.40 mol) of 2-phenoxyethanol and 206.3 g (1.43 mol) of caprylic acid were heated together with 0.4 g of TEGOKAT® 188 (tin(II) oxide; 0.1% by weight based on the total composition) for 5 h at 220° C. on a water separator. Excess acid was distilled off in vacuo and the reaction mixture was cooled. The product was neutralized and deodorized with potassium hydroxide solution. After adding 0.8 g of TEGO® TINEX P, the product was filtered, giving 362.8 g of an odorless oil: OHV=1.2; AV=0.1; color (APHA)=56 Hazen.

### Example 3

### Preparation of 2-phenoxyethyl-2-ethylhexanoic acid ester

96.8 g (0.70 mol) of 2-phenoxyethanol and 103.2 g (0.72 mol) of 2-ethylhexanoic acid were heated together with 1.0 g of para-toluenesulphonic acid for 8 h at 150° C.-160° C. on a water separator. The reaction mixture was cooled and the catalyst was neutralized with potassium hydroxide solution. The product was then bleached and deodorized with hydrogen peroxide. Following filtration, 175.0 g of an odorless oil were obtained: OHV<1; AV=0.2; color (APHA)=40 Hazen.

### Example 4

### Preparation of 2-phenoxyethylcyclohexanecarboxylic acid ester

111.6 g (0.81 mol) of 2-phenoxyethanol and 104.5 g (0.81 mol) of cyclohexanecarboxylic acid were heated together with 1.0 g of para-toluenesulphonic acid for 6 h at 150° C.-160° C. on a water separator. The reaction mixture was cooled and neutralized with potassium hydroxide solution. The product was then deodorized. Following filtration, 192.5 g of an odorless oil were obtained: OHV<1; AV=0.2; color (APHA)=70 Hazen.

### Example 5

### Preparation of 2-phenoxyethyl-2-butyloctanoic acid ester

81.7 g (0.59 mol) of 2-phenoxyethanol and 118.3 g (0.59 mol) of 2-butyloctanoic acid (ISOCARB® 12, Sasol) are heated together with 0.9 g of para-toluenesulphonic acid for 6 h at 150° C.-160° C. on a water separator. The reaction mixture was cooled and neutralized with potassium hydroxide solution. The product was then bleached and deodorized with hydrogen peroxide. Following filtration, 185.3 g of an odorless oil are obtained: OHV<1; AV=0.2; color (APHA)=74 Hazen.

### Example 6

### Preparation of 1-phenoxy-2-propyloctanoic acid ester

106.3 g (0.70 mol) of phenoxypropanol (Bayer) and 103.7 g (0.72 mol) of caprylic acid were heated together with 1.1 g of para-toluenesulphonic acid for 6 h at 150° C.-160° C. on a water separator. The reaction mixture was cooled and neutralized with potassium hydroxide solution. The product was then bleached and deodorized with hydrogen peroxide. Following filtration, 191.0 g of an odorless oil were obtained: OHV<1; AV=0.1; color (APHA)=85 Hazen.

### Example 7

### Preparation of 1-phenoxy-2-propylcyclohexanecarboxylic acid ester

106.3 g (0.70 mol) of phenoxypropanol (Bayer) and 89.7 g (0.70 mol) of cyclohexanecarboxylic acid were heated together with 1.0 g of para-toluenesulphonic acid for 8 h at 150° C.-160° C. on a water separator. The reaction mixture was cooled and neutralized with potassium hydroxide solution. The product was then bleached and deodorized with hydrogen peroxide. Following filtration, 179.9 g of an odorless oil were obtained:

OHV=1.1; AV=0.1; color (APHA)=62 Hazen.

### Example 8

### Dissolving Power of UV Photoprotective Filters

To test the dissolving power of crystalline UV photoprotective filters in phenoxyalkyl esters, four representative UV-A and/or UV-B photoprotective filters were selected by way of representation. These were benzophenone-3 (2-hydroxy-4-methoxybenzophenone, BP-3), Butyl Methoxydibenzoylmethane (BMDM), Octyl Triazone (2,4,6-trianilino-p-(carbo-2′-ethylhexyl-1′-oxy)-1,3,5-triazines, EHT) and Bemotrizinol (bis-ethylhexyloxyphenol meth-oxyphenyl triazine, BEMT).

The dissolving power of conventional ester oils for these compounds was in most cases not satisfactory. A compound with good dissolving power for UV photoprotective filter substances is Tegosoft® TN, which has already been mentioned and which has therefore also been widely established as an ingredient of sunscreen formulations.

To determine the dissolving power for these three UV photoprotective filters, in each case a certain amount (50 g) of one of the compounds according to the invention was initially introduced and heated to 22° C. One (1) percent by weight of a UV photoprotective filter was added and the mixture was stirred until this amount had dissolved completely and homogeneously. This operation was repeated until the maximum dissolvable amount of the UV photoprotective filter was exceeded. For complete dissolution in the case of relatively high concentrations, a prolonged stirring time of several hours was often necessary.

If the maximum concentration had been roughly determined in this way, to finely determine the concentration range around this maximum concentration the procedure was repeated using smaller initial weight amounts of the IN photoprotective filter.

The reference used was the compound Tegosoft® TN (C12-15 alkyl benzoate).

| | Benzophenone- | Tinosorb | | Uvinul |
|---|---|---|---|---|
| Substance name | 3 | S | BMDM | T 150 |
| Tegosoft ® TN | 15% | 9% | 13% | 4% |
| [as comparison] (INCI: | | | | |
| C12-15 Alkyl Benzoate) | | | | |
| 2-Phenoxyethyl caproate | 28% | 11% | 18% | 6% |
| 2-Phenoxyethyl | 30% | 12% | 19% | 6% |
| cyclohexanoate | | | | |
| 2-Phenoxyethyl caprylate | 28% | 11% | 18% | 6% |
| 2-Phenoxyethyl | 25% | 12% | 17% | 4% |
| 2-ethylhexanoate | | | | |
| 2-Phenoxyethyl | 25% | 9% | 16% | 4% |
| isononanoate | | | | |
| 2-Phenoxyethyl | 20% | 9% | 13% | 4% |
| 2-propylheptanoate | | | | |
| 2-Phenoxyethyl | 19% | 8% | 14% | 5% |
| 2-butyloctanoate | | | | |
| 1-Phenoxy-2-propyl | 27% | 10% | 16% | 6% |
| cyclohexanoate | | | | |
| 1-Phenoxy-2-propyl | 21% | 9% | 14% | 6% |
| caprylate | | | | |
| 1-Phenoxy-2-propyl 2- | 21% | 9% | 15% | 4% |
| ethylhexanoate | | | | |

As is clear from the above values, the dissolving power of the compounds according to the invention is significantly better than the dissolving power of Tegosoft® TN.

### Example 9

### Cosmetic Formulations

The compounds of the general formula (I) according to the invention can be used either in oil-in-water or water-in-oil emulsions.

The formulation examples are intended to serve to illustrate, by way of example the ability of e.g., 2-phenoxyethyl caprylate to be used in cosmetic emulsions and do not limit the subject matter of the invention.

Unless noted otherwise, all of the quantitative data in % are parts by weight. Preparation and homogenization steps take place by customary methods.

### Formulation Example A

### O/W Sunscreen Lotion

| Composition | % w/w |
|---|---|
| Polyglyceryl-3 methylglucose Distearate (TEGO ® Care 450, | 3.0 |
| Evonik Goldschmidt GmbH) | |
| Glyceryl Stearate (TEGIN ® M Pellets, Evonik Goldschmidt | 1.0 |
| GmbH) | |
| 2-Phenoxyethyl caprylate | 8.5 |
| Simmondsia Chinensis (Jojoba) Seed Oil (jojoba oil) | 1.5 |
| Ethylhexyl Salicylate | 5.0 |
| Bis-Ethylhexyloxyphenol Methoxyphenyl Triazine | 9.0 |
| Ethylhexyl Methoxycinnamate | 9.0 |
| Butyl Methoxydibenzoylmethane | 3.0 |
| Carbomer (TEGO ® Carbomer 134, Evonik Goldschmidt | 0.2 |
| GmbH) | |
| C12-15 alkyl Benzoate (TEGOSOFT ® TN, Evonik | 1.0 |
| Goldschmidt GmbH) | |
| NaOH (10% strength aqueous solution) | 0.6 |
| Perfume, preservative | q.s. |
| Water dist. | ad 100 |
| pH | 7.2 |
| SPF (Optometrics) | 44 |

### Formulation Example B

### O/W Sunscreen Spray

| Composition | % w/w |
|---|---|
| 2-Phenoxyethyl caprylate | 5.6 |
| Decyl Cocoate (TEGOSOFT ® DC, Evonik Goldschmidt | 2.5 |
| GmbH) | |
| Tocopheryl Acetate | 0.5 |
| Bis-Ethylhexyloxyphenol Methoxyphenyl Triazine | 3.0 |
| Ethylhexyl Triazone | 5.0 |
| Octocrylene | 5.0 |
| Butyl Methoxydibenzoylmethane | 3.0 |
| Cetearyl Glucoside (TEGO ® Care CG 90, Evonik | 1.0 |
| Goldschmidt GmbH) | |
| Cetearyl Alcohol (TEGO ® Alkanol 1618, Evonik | 0.5 |
| Goldschmidt GmbH) | |
| Glycerol | 2.0 |
| Carbomer (TEGO ® Carbomer 141, Evonik Goldschmidt | 0.05 |
| GmbH)) | |
| Acrylates/C10-30 alkyl Acrylate Crosspolymer (TEGO ® | 0.05 |
| Carbomer 341 ER, Evonik Goldschmidt GmbH) | |
| Decyl Cocoate (TEGOSOFT ® DC, Evonik Goldschmidt | 0.4 |
| GmbH | |
| NaOH (10% strength aqueous solution) | 0.4 |
| Perfume, preservative | q.s. |
| Water dist. | ad 100 |
| pH | 7.4 |
| SPF Optometrics | 14 |

### Formulation Example C

### O/W Sunscreen Cream

| Composition | % w/w |
|---|---|
| Cetearyl Alcohol (TEGO ® Alkanol 1618, Evonik | 0.5 |
| Goldschmidt GmbH) | |
| Glyceryl Stearate (TEGIN ® M Pellets, Evonik | 0.5 |
| Goldschmidt GmbH) | |
| Octocrylene | 5.0 |
| Ethylhexyl Salicylate | 5.0 |
| Bis-Ethylhexyloxyphenol Methoxyphenyl Triazine | 4.0 |
| Butyl Methoxydibenzoylmethane | 3.0 |
| Ethylhexyl Triazone | 2.0 |
| Ethylhexyl Methoxycinnamate | 3.0 |
| 2-Phenoxyethyl caprylate | 5.0 |
| Xanthan Gum | 0.3 |
| Acrylates/C10-30 alkyl Acrylate Crosspolymer (TEGO | 0.2 |
| Carbomer 341 ER, Evonik Goldschmidt GmbH) | |
| Tocopheryl Acetate | 0.5 |
| Glyceryl Stearate Citrate (AXOL ® C 62, Evonik | 2.5 |
| Goldschmidt GmbH) | |
| Glycerol | 3.0 |
| NaOH (10% strength aqueous solution) | 0.6 |
| Perfume, preservative | q.s. |
| Water dist. | ad 100 |
| pH | 5.5 |
| SPF Optometrics | 50+ |

### Formulation Example D

### Light W/O Sunscreen Lotion

| Composition | % w/w |
|---|---|
| Cetyl PEG/PPG-10/1 Dimethicone (ABIL ® EM 90, | 2.5 |
| Evonik Goldschmidt GmbH) | |
| Diethylhexyl Carbonate (TEGOSOFT ® DEC, | 3.5 |
| Evonik Goldschmidt GmbH) | |
| 2-Phenoxyethyl caprylate | 8.0 |
| Beeswax | 0.5 |
| Hydrogenated Castor Oil | 0.5 |
| Tocopheryl Acetate | 0.5 |
| Ethylhexyl Triazone | 3.0 |
| Ethylhexyl Methoxycinnamate | 3.0 |
| Ethylhexyl Salicylate | 4.0 |
| Butyl Methoxydibenzoylmethane | 4.0 |
| Bis-Ethylhexyloxyphenol Methoxyphenyl Triazine | 4.0 |
| Sodium Carboxymethyl Beta-Glucan (GluCare ® S, | 0.2 |
| Evonik Goldschmidt GmbH) | |
| Allantoin | 0.1 |
| Glycerol | 1.0 |
| NaCl | 0.5 |
| Water, dist. | 64.7 |
| Perfume, preservative | q.s. |
| SPF Optometrics | 50+ |

### Formulation Example E

### W/O Sunscreen Lotion

| Composition | % w/w |
|---|---|
| Polyglyceryl-4 Diisostearate/Polyhydroxystearate/Sebacate | 3.0 |
| (ISOLAN ® GPS, Evonik Goldschmidt GmbH) | |
| Hydrogenated Castor Oil | 0.2 |
| Microcrystalline Wax | 0.2 |
| Diethylhexyl Carbonate (TEGOSOFT ® DEC, Evonik | 4.0 |
| Goldschmidt GmbH) | |
| 2-Phenoxyethyl caprylate | 8.0 |
| Octocrylene | 4.0 |
| Ethylhexyl Methoxycinnamate | 2.0 |
| Butyl Methoxydibenzoylmethane | 3.0 |
| Bis-Ethylhexyloxyphenol Methoxyphenyl Triazine | 4.0 |
| Ethylhexyl Triazone | 2.0 |
| Sodium Carboxymethyl Beta-Glucan (GluCare ® S, | 0.1 |
| Evonik Goldschmidt GmbH) | |
| Sodium Hyaluronate (HyaCare ®, Evonik Goldschmidt GmbH) | 0.1 |
| Glycerin | 2.0 |
| Water, dist. | 67.4 |
| Perfume, preservative | q.s. |
| SPF calculated | 31 |

### Formulation Example F

### Antiperspirant Stick

| Composition | % w/w |
|---|---|
| Cyclomethicone | 41.0 |
| Glyceryl Stearate (TEGIN ® M Pellets, Evonik | 1.0 |
| Goldschmidt GmbH) | |
| Stearyl Alcohol (TEGO ® Alkanol 18, Evonik | 18.0 |
| Goldschmidt GmbH) | |
| Hydrogenated Castor Oil | 2.5 |
| 2-Phenoxyethyl caprylate | 5.0 |
| C12-15 alkyl Benzoate (TEGOSOFT ® TN, Evonik | 4.0 |
| Goldschmidt GmbH) | |
| Diethylhexyl Carbonate (TEGOSOFT ® DEC, Evonik | 5.0 |
| Goldschmidt GmbH) | |
| Aluminium Zirconium Tetrachloro-hydrex GLY | 20.0 |
| Talc | 3.0 |
| Silica | 0.5 |

### Formulation Example G

### 24 h AP/Deo Stick

| Composition | % w/w |
|---|---|
| PPG-3 Myristyl Ether (TEGOSOFT ® APM, Evonik | 5.0 |
| Goldschmidt GmbH) | |
| 2-Phenoxyethyl caprylate | 5.0 |
| Stearyl Alcohol (TEGO ® Alkanol 18, Evonik Goldschmidt | 16.25 |
| GmbH) | |
| Hydrogenated Castor Oil | 1.75 |
| Cyclomethicone | 44.5 |
| Silica Dimethyl Silylate (AEROSIL ® R 972 V, Evonik | 3.0 |
| Degussa GmbH) | |
| Aluminium Chlorohydrate (50%) | 20.0 |
| Polyglyceryl-3 Caprylate (TEGO ® Cosmo P 813, Evonik | 0.5 |
| Goldschmidt GmbH) | |
| Zinc Ricinoleate, Lysine, Propylene Glycol (TEGODEO ® | 4.0 |
| LYS, Evonik Goldschmidt GmbH) | |

### Formulation Example H

### PEG-Free O/W Antiperspirant Lotion

| Composition | % w/w |
|---|---|
| Methyl Glucose Sesquistearate (TEGO ® Care PS, Evonik | 1.75 |
| Goldschmidt GmbH | |
| Polyglyceryl-4 Laurate (TEGO ® Care PL 4, Evonik | 0.25 |
| Goldschmidt GmbH) | |
| 2-Phenoxyethyl caprylate | 3.0 |
| Diethylhexyl Carbonate (TEGOSOFT ® DEC, Evonik | 2.0 |
| Goldschmidt GmbH) | |
| PPG-14 Butyl Ether (TEGOSOFT ® PBE, Evonik | 2.0 |
| Goldschmidt GmbH) | |
| Polyglyceryl-3 Caprylate (TEGO ® Cosmo P 813, Evonik | 0.5 |
| Goldschmidt GmbH) | |
| Water, dist. | 74.5 |
| Hydroxyethyl Cellulose | 1.0 |
| Aluminium Chlorohydrate (50%) | 15.0 |
| Perfume | q.s. |

### Formulation Example J

Deodorizing O/W Hand and Foot Cream

| Composition | % w/w |
|---|---|
| Ceteareth-25 (TEGINACID ® C, Evonik Goldschmidt GmbH) | 2.0 |
| Glyceryl Stearate (TEGIN ® M Pellets, Evonik Goldschmidt | 4.0 |
| GmbH) | |
| Stearyl Alcohol (TEGO ® Alkanol 18, Evonik Goldschmidt | 2.5 |
| GmbH) | |
| Cetearyl Isononanoate (TEGOSOFT ® CI, Evonik Goldschmidt | 2.5 |
| GmbH) | |
| 2-Phenoxyethyl caprylate | 4.0 |
| Zinc Ricinoleate (TEGODEO ® PY 88, Evonik Goldschmidt | 2.0 |
| GmbH) | |
| Glycerol | 3.0 |
| Water, dist. | 79.0 |
| Carbomer (TEGO ® Carbomer 134, Evonik Goldschmidt | 0.2 |
| GmbH) | |
| Cetearyl Isononanoate (TEGOSOFT ® CI, Evonik Goldschmidt | 0.8 |
| GmbH) | |

### Formulation Example K

### O/W Antiperspirant Roll-On

| Composition | % w/w |
|---|---|
| Steareth-2 (TEGO ® Alkanol S 2 Pellets, Evonik | 2.2 |
| Goldschmidt GmbH) | |
| Steareth-20 (TEGO ® Alkanol S 20 P, Evonik | 1.0 |
| Goldschmidt GmbH) | |
| Cetearyl Ethylhexanoate (TEGOSOFT ® liquid, Evonik | 2.0 |
| Goldschmidt GmbH) | |
| 2-Phenoxyethyl caprylate | 2.0 |
| Dimethicone (ABIL ® 350, Evonik Goldschmidt GmbH) | 0.5 |
| Glycerol | 3.0 |
| Water, dist. | 58.8 |
| Triethylcitrate | 0.5 |
| Aluminium Chlorohydrate (50%) | 30.0 |

While the present invention has been particularly shown and described with respect to preferred embodiments thereof, it will be understood by those skilled in the art that the foregoing and other changes in forms and details may be made without departing from the spirity and scope of the present invention. It is therefore intended that the present invention not be limited to the exact forms and details described and illustrated, but fall within the scope of the appended claims.

What is claimed is:

1. A formulation comprising at least one phenoxyalkyl ester of general formula (I) 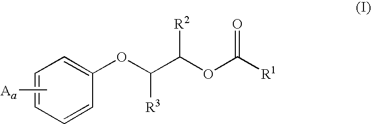
where R¹ is an unbranched or branched, saturated or unsaturated C₅₋₁₁ alkyl or cycloalkyl radical,
R² and R³, independently of one another, identical or different, are hydrogen or methyl and
Aₐ where a=1-5, independently of one another, identical or different, are selected from the group: H, F, Cl, alkyl or fluoroalkyl radical, CN, CO₂R⁴, OH, OR⁵, O₂CR⁶, NR⁷R⁸, NO₂, SO₃R⁹, where R⁴ to R⁹, independently of one another, identical or different, are selected from the group: H, alkyl or fluoroalkyl radical.

2. The formulation according to claim 1, further comprising
(a) 0.1-60 percent by weight of the at least one phenoxyalkyl ester of the general formula (I),
(b) 0.1-20 percent by weight of a surfactant and/or emulsifier and/or coemulsifier,
(c) 0.1-40 percent by weight of oil bodies, and
(d) 0-98 percent by weight of water,
where the percentages by weight of components (a), (b), (c) and (d) add up to 100 percent by weight.

3. The formulation according to claim 1, wherein the group OOC—R¹ of the at least one phenoxyalkyl ester is selected from the group:
caproic acid, cyclopentanecarboxylic acid, 2-methylpentanoic acid, heptanoic acid, cyclohexanecarboxylic acid, caprylic acid, 2-ethylhexanoic acid, sorbic acid, pelargonic acid, isononanoic acid, 3,5,5-trimethylhexanoic acid, capric acid, 2-propylheptanoic acid, iso-decanoic acid, undecanoic acid, undecylenic acid and 2-butyloctanoic acid, and mixtures comprising at least two of these carboxylic acids.

4. The formulation according to claim 1, wherein R² and R³ of the at least one phenoxyalkyl ester are selected from the group:
R²═R³═H,
R²═H, R³=methyl,
R²=methyl, R³═H,
and combinations of these.

5. The formulation according to claim 1, wherein the at least one phenoxyalkyl ester is a 2-phenoxyethylhexanoic acid ester, 2-phenoxyethylheptanoic acid ester, 2-phenoxyethyloctanoic acid ester, 2-phenoxyethyl-2-ethylhexanoic acid ester, 2-phenoxyethylsorbic acid ester, 2-phenoxyethylpelargonic acid ester, 2-phenoxyethylcyclo-pentanecarboxylic acid ester, 2-phenoxyethylcyclohexancarboxylic acid ester, 2-phenoxy-ethylisononanoic acid ester, 2-phenoxyethyl-3,5,5-trimethylhexanoic acid ester, 2-phenoxy-ethylisodecanoic acid ester, 2-phenoxyethylundecanoic acid ester, 2-phenoxyethyl-2-butyl-octanoic acid ester, 1-phenoxy-2-propyloctanoic acid ester, 1-phenoxy-2-propyl-2-ethyl-hexanoic acid ester, 1-phenoxy-2-propylpelargonic acid ester, 1-phenoxy-2-propylcyclo-hexanecarboxylic acid ester, 1-phenoxy-2-propylisononanoic acid ester, 1-phenoxy-2-propyl-3,5,5-trimethylhexanoic acid ester, 1-phenoxy-2-propyl-isodecanoic acid ester or 1-phenoxy-2-propyl-2-butyloctanoic acid ester.

6. The formulation according to claim 1, wherein the formulation is a sunscreen preparation including at least one sunscreen component.

7. The formulation according to claim 1, wherein the at least one phenoxyalkyl ester of formula (I) is used as solubility promoter or solvent for at least one UV photoprotective filter substance.

8. The formulation according to claim 7, wherein the at least one UV photoprotective filter substance is a triazine derivative.

9. The formulation according to claim 7, wherein the at least one UV photoprotective filter substance is selected from the group of compounds consisting of 2-ethylhexyl 2-cyano-3-phenylcinnamate, 2,4-bis{[4-(2-ethylhexyloxy)-2-hydroxy]phenyl}-6-(4-methoxyphenyl)-1,3,5-triazine, dioctylbutylamidotriazone, 2-hydroxy-4-methoxybenzophenone, 2-hydroxy-4-methoxy-4′-methylbenzophenone, 2,2′-dihydroxy-4-methoxybenzophenone, di-2-ethylhexyl 4-methoxybenzmalonate, 2,4,6-tris[anilino(p-carbo-2′-ethyl-1′-hexyloxy)]-1,3,5-triazine, 2,4-bis[5,1-(dimethylpropyl)benzoxazol-2-yl-(4-phenyl)-imino]-6-(2-ethylhexyl)-imino-1,3,5-triazine, 2,4-bis{[4-(2-ethylhexyloxy)-2-hydroxy]phenyl}-6-(4-methoxyphenyl)-1,3,5-triazine and 2-[4,6-bis(2,4-dimethylphenyl)-1,3,5-triazin-2-yl]-5-(octyloxy)phenol.

10. The formulation according to claim 1, wherein the formulation is a deodorant/antiperspirant preparation, and said at least one phenoxyalkyl ester of the formula (I) is used as solubility promoter or solvent of at least one deodorant or antiperspirant active ingredient.

11. The formulation according to claim 10, wherein the at least one deodorant or antiperspirant active ingredient is an aluminium salt, zinc salt or aluminium-zirconium complex.

12. The formulation according to claim 1, wherein the formulation is selected from a cosmetic formulation including at least one cosmetic ingredient, a dermatological formulation including at least one dermatological ingredient and a pharmaceutical formulation including at least one pharmaceutical ingredient.

13. A method for the preparation of a formulation comprising:
adding at least one phenoxyalkyl ester of general formula (I) 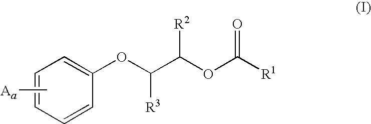
where R¹ is an unbranched or branched, saturated or unsaturated C₅₋₁₁ alkyl or cycloalkyl radical,
R² and R³, independently of one another, identical or different, are hydrogen or methyl and
Aₐ where a=1-5, independently of one another, identical or different, are selected from the group: H, F, Cl, alkyl or fluoroalkyl radical, CN, CO₂R⁴, OH, OR⁵, O₂CR⁶, NR⁷R⁸, NO₂, SO₃R⁹, where R⁴ to R⁹, independently of one another, identical or different, are selected from the group: H, alkyl or fluoroalkyl radical, to at least one component selected from emollients, emulsifiers and surfactants, thickeners/viscosity regulators/stabilizers, UV photoprotective filters, antioxidants and vitamins, hydrotropes, solids and fillers, film formers, pearlescent additives, deodorant and antiperspirant active ingredients, insect repellents, self-tanning agents, preservatives, conditioners, perfumes, dyes, biogenic active ingredients, care additives, superfatting agents, and solvents.

14. The method according to claim 13, wherein the group OOC—R¹ of the at least one phenoxyalkyl ester is selected from the group:
caproic acid, cyclopentanecarboxylic acid, 2-methylpentanoic acid, heptanoic acid, cyclohexanecarboxylic acid, caprylic acid, 2-ethylhexanoic acid, sorbic acid, pelargonic acid, isononanoic acid, 3,5,5-trimethylhexanoic acid, capric acid, 2-propylheptanoic acid, iso-decanoic acid, undecanoic acid, undecylenic acid and 2-butyloctanoic acid, and mixtures comprising at least two of these carboxylic acids.

15. The method according to claim 13, wherein R² and R³ of the at least one phenoxyalkyl ester are selected from the group:
R²═R³═H,
R²═H, R³=methyl,
R²=methyl, R³═H,
and combinations of these.

16. The method according to claim 13, wherein the at least one phenoxyalkyl ester is a 2-phenoxyethylhexanoic acid ester, 2-phenoxyethylheptanoic acid ester, 2-phenoxyethyloctanoic acid ester, 2-phenoxyethyl-2-ethylhexanoic acid ester, 2-phenoxyethylsorbic acid ester, 2-phenoxyethylpelargonic acid ester, 2-phenoxyethylcyclo-pentanecarboxylic acid ester, 2-phenoxyethylcyclohexan carboxylic acid ester, 2-phenoxy-ethylisononanoic acid ester, 2-phenoxyethyl-3,5,5-trimethylhexanoic acid ester, 2-phenoxy-ethylisodecanoic acid ester, 2-phenoxyethylundecanoic acid ester, 2-phenoxyethyl-2-butyl-octanoic acid ester, 1-phenoxy-2-propyloctanoic acid ester, 1-phenoxy-2-propyl-2-ethyl-hexanoic acid ester, 1-phenoxy-2-propylpelargonic acid ester, 1-phenoxy-2-propylcyclo-hexanecarboxylic acid ester, 1-phenoxy-2-propylisononanoic acid ester, 1-phenoxy-2-propyl-3,5,5-trimethylhexanoic acid ester, 1-phenoxy-2-propyl-isodecanoic acid ester or 1-phenoxy-2-propyl-2-butyloctanoic acid ester.
